(19) United States
(12) Patent Application Publication
(10) Pub. No.: US 2004/0175330 A1
(43) Pub. Date: Sep. 9, 2004

(54) Protein chip for diagnosis allergy and detection method for allergen and antibody

(76) Inventor: Geun-Woong Noh, Seoul (KR)

Correspondence Address: GREENBLUM & BERNSTEIN, P.L.C., 1950 ROLAND CLARKE PLACE, RESTON, VA, 20191, US

(21) Appl. No.: 10/482,167

(22) PCT Filed: Aug. 20, 2001
(86) PCT No.: PCT/KR01/01404

(30) Foreign Application Priority Data
Jul. 7, 2001 (KR) 2001/40678

(51) Int. Cl. A61K 49/00

(52) U.S. Cl. 424/9.81

(57) ABSTRACT

This invention is related to the protein chip for allergy diagnosis and the detection method for allergen antibodies. The protein chip of this invention is a solid chip with one crude protein attached to each spot, isolated from various kinds of allergen. By reacting those plates with the sample of allergic patients, the various allergen can be detected at the same time, and the kinds of antibodies related with the allergic reactions caused by the certain allergens.

### BACKGROUND OF THE INVENTION

1. Field of the Invention

This invention is related to the protein chip for allergy diagnosis and the detection method for allergy inducing antibodies.

The immune system in the human body produces antibodies to remove antigens, such as foreign substances, introduced from the outside. The allergy is the symptoms which are caused by antibodies produced against the foreign substances, not harmful for most people, as a result of the hypersensitive immune reaction between antigens and antibodies. As a response to the allergy inducing antigens introduced into human body, antibodies are produced in quantities, such as the IgE inducing allergy, IgG4, known to suppress allergy, and IgG, known to induce general immune reaction. As a result of interaction between these antibodies, bronchial contraction and capillary dilation are induced, and the secreting gland is stimulated, then followed by the symptoms of allergy such as coughing and a runny nose. The disorders caused by allergy are bronchial asthma, allergic rhinitis, allergic conjunctivitis, atopic dernatitis, and contact dernatitis.

One of the therapeutic methods is medication with antihistamine, which is not only effective for temporary relief, but also responsible for the risk of side effects. The most desirable treatment method is strengthening immune function and blocking allergenic substance. Therefore, the detection of allergenic substance is most important for the complete cure of allergy.

The conventional detection methods for allergy induction are those such as the skin reaction test, the induction test, and the blood test. The skin reaction test is the test which is conducted by reacting the allergen with the skin to see whether or not an allergic reaction take a place. Although this method is simple, sensitive and used widely, this methods has some limits, such as the inhibition of the skin reaction for patients with severe eczema, medicated with antihistamine interfere with an accurate quantitative reaction, which is important for efficient treatment.

The induction test is done by reacting the allergen to a patient's eye, nose and bronchus by direct application. This method is used with limitation because of the burden on the patients.

The blood test is the methods measuring the number of total eosinophil or IgE values in the whole blood. Particularly, the FAST (Fluoroallergosorbent test) is used for the measurement of quantitative and qualitative analysis regardless of the status of the patient's skin and medication. But this method is limited to the testing of certain kinds of antibodies, by less sensitivity compared to the skin reaction test, and by its expensiveness.

On the other hand, the protein chips developed actively are in the spot light as a technique for the detection of disease related protein.

In general, most of the protein chips are solid plates with one protein attached in one spot on the plate. But, in the case of allergy, which can be induced by the crude proteins contained in the allergen, the detection of a single protein is of no use for the detection of allergen.

The conventional method involves testing allergy induction by a single allergen, thus there is no way of finding all the causes of allergy induction overall. Also, the conventional method does not provide the way for detecting the specific antibody responsible for the allergy induced by certain allergen among various antibodies. Therefore, for the effective diagnosis of allergy, detection methods are required which involve detecting the various kinds of allergens at one time, testing the antibodies related with an allergic reaction and determining the exact cause.

2. Background and Related Disclosures

The purpose of this invention is to provide the protein chip to detect the cause of the patient's allergy, and to determine the type of antibody related with allergic reaction caused by certain allergen.

Another purpose of this invention is to provide the detection method for allergen and antibodies using the above protein chip.

### SUMMARY OF THE INVENTION

This invention is related to the protein chip for allergy diagnosis and detecting allergen and antibodies. The protein chip of this invention is a solid chip with one crude protein attached in each spot, isolated from various kinds of allergen. By reacting the plate with the sample of allergic patients, the various allergen can be detected at the same time, and the type of the antibody related with the allergic reactions caused by the certain allergens.

### DETAILED DESCRIPTION OF THE INVENTION

This invention provides the protein chip for diagnosing allergy and the detection method for allergen and antibodies. The protein chip of this invention is a solid chip made by attaching crude protein, isolated from various kinds of allergen, to each spot on the protein chip. The protein chip made as described above is employed to react with samples, such as the patient's blood and body fluids, in order to detect the allergen and to determine the type of antibody related with allergic reaction induced by the particular allergen. The crude protein of this invention attached to protein chips, are isolated from allergenic organic material such as house dust, mites, pollen, animal hair, meat including microbes, beans, grains, hardy crops, seafoods, mushrooms, vegetables, fruits, dairy goods, and egg.

Since the all the allergenic proteins could include not only pure proteins, but also non-proteinaceous nitrogen compounds, crude proteins containing all the proteins present in one allergen, are spotted in one point on the solid plate in this invention for the accuracy of the diagnostic test. The crude proteins of this invention attached to the protein chips are the mixed proteins dissolved in the neutral salt solution, ethanol, weak acid and PBS buffer (phosphate buffered saline). By doing this, the protein chips in this invention can contain all the crude proteins present in the allergens spotted in one point on the protein chip.

The crude proteins of this invention, attached to protein chips, are prepared in processed and digested form, as well as fresh, separately. Food-related allergy can be induced not only by fresh food, but also by the cooked and digested form in the body. Therefore the proteins isolated from fresh food, and food in the food allergy inducible form processed by biochemical procedure, such as heating and treatment with artificial, gastric acid and digested form, are used as the crude proteins of this invention attached to protein chips.

The food treatment process with artificial, gastric acid was done as following. The fresh food is mixed with artificial, gastric acid comprising pepsin 0.842 mg/ml, sodium choloride 2.0 mg/ml, pH 1.2 in 1:200 (w/w) ratio, and treated for 30 min at 37.5° C. after adding HCl. To stop the reaction with HCl, the same amount of 10M NaOH as HCl was added and treated for 10 min at 99° C. Trypsin 50 mg/ml was added in a 1:200 ratio (treated antigen: trypsin) and treated in 37.5° C. To stop the reaction, the reactant was treated for 10 min, at 99° C.

The process of isolating the crude proteins from the allergen was done as following.

The reactant was freeze-dried, powdered and treated with hexane, after which the pellet was collected and finally dried. The lipid was removed by repeating the hexane treatment two times.

The powder mixed with a neutral salt solution of 0.5M NaCl was eluted and centrifuged. The upper layer was collected, dialyzed with distilled water and freeze-dried to get the protein to dissolve in ethanol.

The pellet separated from the upper layer in the procedure above was eluted after adding distilled water, centrifuged and collected, the residue removed. This was eluted after mixing it with 1% acetic acid, and then centrifuged. The upper layer was collected, dialyzed with distilled water and collect the proteins dissolved in weak acid.

The pellet separated from the upper layer in the procedure above was eluted after adding distilled water, centrifuged and collected, the residue removed. This was eluted after mixing it with PBS buffer, and then centrifuged. The upper layer was collected and dialyzed with distilled water, after which the proteins dissolved in PBS buffer were collected.

The mixed proteins isolated in the procedure above were employed as the crude proteins in this invention. The crude proteins were quantified with Bradford assay, and dispensed to each well. A certain amount of the crude protein per each food was spotted into each point of the solid plate.

The material of plate suitable for solid protein chip can be glass, commonly used polymer or gel such as modified silicon, tetrafluoroethylene, polystyrene or polyprophylene. For easy attachment of the protein, the surface of the plate can be treated with polymer, plastic, resin, carbohydrate, silica, silica derivatives, carbon, metal, inorganic glass, or membrane. The plate plays a role, not only as a support for fixing proteins, but also as a place for the reaction between the fixed protein antigen and the antibody in the sample. The size of the plate above and the position, size, and shape of the protein fixed in the plate, can be varied according to the purpose of the analysis, and by instruments, such as a spotting machine and a scanner.

The allergen detection methods in this invention detect the antigen-antibody reaction by reacting the crude protein of the protein chip with the samples such as blood or body fluids. The methods for detecting the antibodies related with the allergy reaction induced by certain allergen are the same as the allergen detection methods. Since different colors of chromophore can be used in this process for different kinds of the antibodies, the different kinds of the antibodies can be detected by utilizing different colors of the fluorescence at the same time.

For the interpretation of the result obtained by reacting the protein chip and samples, the secondary antibodies, or tertiary antibodies when necessary, can be utilized which bind to the antibodies specifically, and are combined with identification tags. The identification tag can be an enzyme generating chromophore, radioactive isotope, or fluorescence molecule. The highly sensitive, safe fluorescence molecule is preferred, compared to the enzyme with low sensitivity, and radioactive isotope dangerous for environmental and biomedical reason. The fluorescence molecule appropriate in this invention can be conventional fluorescence dyes such as Cy3, Cy5, FITC, or TRITC. The strength of the fluorescence dye can be determined by the scanner at the appropriate wavelength.

Since the protein chip in this invention determines the cause of the allergy among the many kinds of allergen and the kinds of antibodies related with allergy reaction induced by particular allergen, it can be very helpful towards saving time and effort in allergy diagnosis.

Because the protein chip in this invention requires only a small amount of the sample for the test, the burden on the patient is minimum. The test can be easily performed on children and infants, which was previously difficult. Formerly, a minimum of 20-100 μl blood samples were required for one kind of antibody. Utilizing the protein chip of this invention, a 60 μl blood sample is enough to examine a maximum of 5000 antibodies at the same time. For the examination of two kinds of antibodies, an only 1/3300-1/12000 volume is enough, helpful towards minimizing the sample volume dramatically compared to existing tests.

The protein chip of this invention is helpful for the effective treatment of allergies, which can detect not only major antibodies reacted to major allergen contained in the allergen, but also antibodies reacted to the minor antigens.

The protein chip of this invention makes it possible to analyze the exact status of allergy induction in the human body and the real cause of the allergy, including not only fresh food itself, but also the product obtained from processing it with heat and artificial, gastric acid for food intake.

This invention is described in the examples below in detail. However, this invention is not limited to these examples.

### BRIEF DESCRIPTION OF THE DRAWINGS

FIG. 1 is the SDS PAGE result of the crude proteins isolated from milk, egg white, egg yolk, soy bean, fresh garlic, garlic cooked with artificial, gastric acid, unmilled rice, and adlay, stained with coomasi blue. (M: Molecular marker, Lane 1: Positive control (human IgE), Lane 2: milk, Lane 3: egg white, Lane 4: egg yolk, Lane 5: soy bean, Lane 6: fresh garlic, Lane 7: cooked garlic, Lane 8: unmilled rice, Lane 9: adlay.)

FIG. 2 is the western blot result of crude proteins isolated from milk, egg white, egg yolk, soy bean, fresh garlic, garlic cooked with artificial, gastric acid, unmilled rice, and adlay, reacted with the blood samples of allergic dermatitis, (Lane 1: Negative control (HSA), Lane 2: Positive control (human IgE), Lane 3: milk, Lane 4: egg white, Lane 5: egg yolk, Lane 6: soy bean, Lane 7: fresh garlic, Lane 8: cooked garlic, Lane 9: unmilled rice, Lane 10: adlay)

### DETAILED DESCRIPTION OF THE PREFERRED EMBODIMENTS

### EXAMPLE 1

### Production of the Protein Chip

1) Isolation of Crude Protein from Allergy Inducing Foods

To isolate the crude proteins from the allergy inducing foods, 150 g each of milk, egg white, egg yolk, soy bean, fresh garlic, cooked-garlic processed with artificial, gastric acid, unmilled rice, and adlay were prepared. The foods were freeze-dried for more than 30 hours to remove water, and then grinded into powder with a grinder. Hexane was added into the dried samples in a 1:5 (w/v) ratio, stirred for 30 minutes, and allowed to sit for 30 minutes. The upper layer was decanted and the bottom layer was dried. The hexane treatment process was repeated two times to remove the lipid.

The powder without lipid was dissolved in 0.5M NaCl solution in a 1:4 (w/v) ratio, eluted for 3 hours at 4° C. and centrifuged at 15,000 rpm at 4° C. for 1 hour. The upper layer obtained was dialyzed with distilled water for 24 hours, and freeze-dried. The protein dissolved in neutral salt solution was obtained.

The pellet separated from the upper layer during the last step was mixed with distilled water, eluted for 1 hour, and centrifuged for 1 hour at 15000 rpm. The obtained pellet was mixed with 70% ethanol in a 1:4 (w/v) ratio, stirred for 3 hours at 4° C., and centrifuged at 15,000 rpm at 4° C. for 1 hour. The obtained upper layer was dialyzed with distilled water for 24 hours, and freeze-dried. The protein dissolved in ethanol was obtained.

The pellet separated from the upper layer during the last step was mixed with distilled water, eluted for 1 hour, and centrifuged for 1 hour at 15000 rpm. The obtained pellet was mixed with 1% acetic acid in a 1:4 (w/v) ratio, stirred for 3 hours at 4° C., and centrifuged at 15,000 rpm at 4° C. for 1 hour. The obtained upper layer was dialyzed with distilled water for 24 hours and freeze-dried. The protein dissolved in weak acid was obtained.

The pellet separated from the upper layer during the last step was mixed with distilled water, eluted for 1 hour, and centrifuged for 1 hour at 15000 rpm. The obtained pellet was mixed with PBS solution in a 1:4 (w/v) ratio, stirred for 3 hours at 4° C., and centrifuged at 15,000 rpm at 4° C. for 1 hour. The obtained upper layer was dialyzed with distilled water for 24 hours, freeze-dried and the protein dissolved in PBS solution was obtained.

The crude protein was obtained by mixing all the proteins obtained from each step.

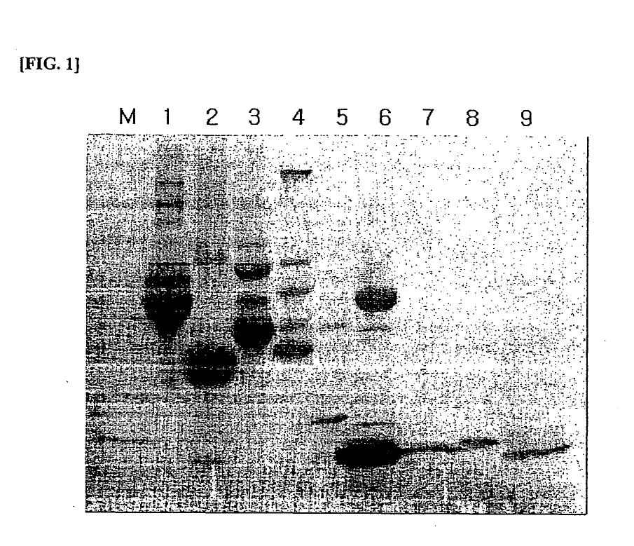

The crude protein was quantified with Bradford assay. The standard curve was obtained by using BSA (Bovine Serum Albumin). The color-reaction was employed for each crude protein, and the absorbance was measured. The concentration of crude protein was determined by finding the matching concentration with each absorbance on the standard curve. The concentration of crude proteins was adjusted into 10 μg/ml. SDS-PAGE (sodium dodecyl sulfate-polyacrylamide gel electrophoresis) was proceeded on 4-20% Tris-glycine precasting gel with 125 V for 90 minutes. The gel done with electrophoresis was stained overnight with coommassie blue solution, and destained with solution of 50% methanol, 10% acetic acid, and 40% water. The good isolation of crude protein from each food was determined (FIG. 1). For garlic, it was checked that cooked garlic lost much of the crude protein in comparison to fresh garlic.

2) Production of the Protein Chip in this Invention

To produce the protein chip, the crude proteins (10 μg/ml) isolated from each of the foods were dispensed into the 384 wells. Cartesian MicroSys 4100 was employed as a spotting machine. The protein chip was produced by spotting 10 nl of the crude proteins diluted on the surface of the slides, according to array order described in FIG. 1.

TABLE 1

| | Negative | Positive | | Egg | | | Fresh | Cooked | Unmilled | |
|---|---|---|---|---|---|---|---|---|---|---|
| | control | control | Milk | white | Egg yolk | Soy bean | garlic | garlic | rice | adlay |
| array | undiluted | undiluted | undiluted | undiluted | undiluted | undiluted | undiluted | undiluted | undiluted | undiluted |
| | 1/10 | 1/10 | 1/10 | 1/10 | 1/10 | 1/10 | 1/10 | 1/10 | 1/10 | 1/10 |
| | 1/100 | 1/100 | 1/100 | 1/100 | 1/100 | 1/100 | 1/100 | 1/100 | 1/100 | 1/100 |

### EXAMPLE 2

### Detection of the Allergen Inducing Food by Use of the Protein Chip of this Invention

To detect the allergen inducing food by using Protein chip of this invention, the protein chip of this invention was allowed to react with TBS-T (Tris Buffered Saline—0.1% Tween 20) with 1% BSA to prevent nonspecific reaction. After washing in TBS-T solution for 15 minute, 3 times, The protein chips were allowed to react with a blood sample, 60 μl, for 1 hour. After washing, biotin-bound anti-human IgE (1:1000 diluted, Vector Co.) was allowed to react for 1 hour at 37° C. To read the protein chip, ArrayWoRx (Applied Precision Co) was employed to read the Cy3 signal, and Imagene (Biodiscovery Co) was used as a program. The result was disclosed in Table 2 below.

TABLE 2

| | Intensity of fluoresnece | | | |
|---|---|---|---|---|
| food | Undiluted | 1/10 diluted | 1/100 diluted | Result |
| Negative | — | 5635.86 | — | negative |
| control | | | | |
| Positive | — | 45811.78 | — | positive |
| control | | | | |
| milk | 7640.71 | 9529.38 | 9588.97 | negative |
| Egg white | 120500.48 | 9166.24 | 7117.69 | positive |
| Egg yolk | 21244.61 | 11595.51 | 6269.52 | positive |
| Soy bean | 13048.82 | 12232.13 | 8516.96 | positive |
| Fresh garlic | 30501.29 | 18947.53 | 8333.95 | positive |
| Cooked garlic | 5932.04 | 9061.51 | 5635.86 | negative |
| Unmilled rice | 9935.57 | 10069.98 | 8406.52 | negative |

As shown at Table 2, it was determined that antibodies against the egg white, egg yolk, soybean, fresh garlic, and adlay were present in the patient's blood.

### COMPARATIVE EXAMPLE

### Detection of Allergen Inducing Foods by Conventional Methods

1) Detection of Allergen Inducing Foods by FAST Test

The FAST test imported from abroad was done for the patient of example 2 on milk, egg white, egg yolk, soybean, fresh garlic, rice, and barley. (Table 3)

TABLE 3

| | | | Cooked | Fresh | | | | |
|---|---|---|---|---|---|---|---|---|
| foods | barley | Rice | garlic | garlic | soybean | Egg yolk | Eff whilte | milk |
| Measurement | 66 | 45 | — | 121 | 63 | 11 | 8 | 20 |
| result | positive | Negative | — | positive | positive | negative | negative | negative |

As shown at table 3, the patient demonstrated a positive reaction against soybean, garlic, and barley. While strong positive result against all the garlic were seen, the result for cooked rice could not be determined by this method. Only strong positive reaction against whole food of garlic was shown.

2) Allergen Test of Foods by Western Analysis

Western analysis which can be utilized for antigen-antibody reaction were employed to test the presence of antibody positive for the antigen for patient of example 2.

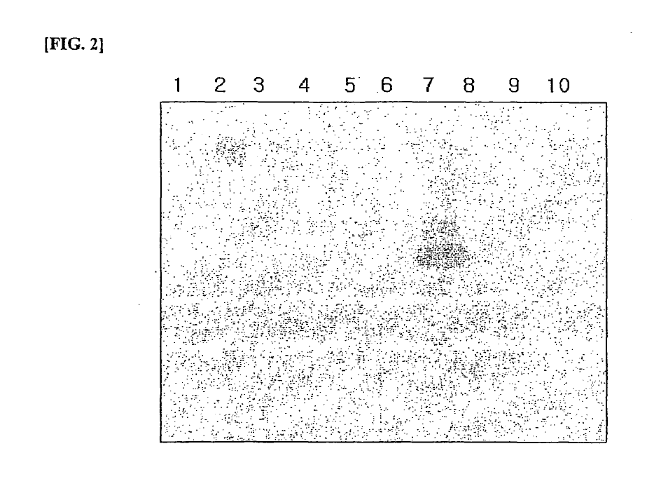

With the same method as in example 1, crude protein isolated from patient was run on electrophoresis gel and gel was transferred to the NC(Nitrocellulose) membrane, 16V for 3 hour 30 minute. The NC (Nitrocellulose) membrane containing the crude protein was reacted with PBS buffer containing 0.5% skim milk for 30 minutes to prevent nonspecific reaction. The blood sample isolated from the patient was diluted at a 1:3 ratio, and reacted for 1 hour with biotin-bound anti-human IgE (1:1000 diluted, Vector Co.). In each step, the membrane was washed with PBS-T (Phosphate Buffered Saline—0.1% Tween 20), for 15 minutes 3 times, and reacted with streptavidin (1:1000 diluted, Caltag Co) for 1 hour. The membrane was washed for 15 minutes and employed TMB (tetramethylbenzadin) staining solution to check the staining-status. (FIG. 2)

As it is shown in the table 2, the presence of the antibody against egg yolk and fresh garlic was checked in the patients above. Very weak positive reaction was seen in the soybean, cooked garlic, and adlay. For the cooked garlic, there was no response to the allergy related antibody, which is different from fresh garlic. This result was consistent with the disappearence of antigenic crude protein in the cooked garlic as shown in the FIG. 1.

In Example 2 and the comparative example, the egg white is determined to be negative according to the FAST analysis and western analysis. However, analysis using the protein chips of this invention showed a positive reaction. Therefore, the protein chip of this reaction has advantages such as excellent sensitiveness and precise diagnosis of allergy compared to the conventional diagnosis reagent.

According to the diagnosis result of garlic, conventional diagnosis methods, such as FAST, were not good enough to distinguish the fresh and cooked one.

### Example 3

### Detection of the Type of the Antibody Against Particular Food by Using the Protein Chip of this Invention

To detect the type of the antibody against particular foods by using the protein chip of this invention, the secondary antibody containing different kinds of fluorescence for different antibodies was utilized. Cy3 and FITC were used as fluorescence according to the different kinds of the secondary antibody. The intensity of the fluorescence was measured at wavelengths 543 nm and 488 nm.

Since the result were obtained at the different wavelengths depending on the kinds of the antibody, it is possible to determine different kinds of antibodies at the same time.

### INDUSTRIAL APPLICABILITY

The protein chip of this invention has several advantage. Since, by using this protein chip, determining several kinds of allergen at the same time is possible with small amounts of the sample, it is easy and quick to test for specific antibodies related with allergy reaction by particular allergen, the effect of this protein chip is economical without the burden of patient.

The protein chip of this invention is so precise that it can be applied to detect not only the antibody reacting against main antibody, but also the antibody reacting against the minor antigen. Since this protein chip can be applied to examine not only fresh food, but also processed foods for intake, it is useful to suggest the guidelines for treatment by understanding the cause of the allergy. Also, the allergy patients can intake the foods safely by choosing the appropriate, processed form.

By using the protein chip of this invention, which can be applied to examining various forms of the foods ingested by Korean patients, accurate diagnosis can be expected without misdiagnosis by using the conventional imported diagnosis reagent.

By using protein chip of this invention, allergy diagnosis can be automated and can make a profile of the protein related with each allergy into a database for each of the individuals and groups. The allergy-inducing model, according to the various foods can be built, which can be utilized for public health from a national point of view.

What is claimed is:

1. The protein chip for allergy diagnosis, comprising the step of attaching each crude protein, isolated from various kinds of allergen, in one spot on the solid chip.

2. The protein chip for allergy diagnosis, according to the claim 1, wherein said crude protein are prepared by isolating the proteins dissolved in neutral salt solution, ethanol, weak acid, or PBS buffer and mixing those.

3. The protein chip for allergy diagnosis, according to the claim 1, wherein said crude protein are prepared by isolating the proteins from the fresh foods, or processed food by heating or treatment with the artificial, gastric juice.

4. The protein chip for allergy diagnosis, according to the claim 3, wherein said artificial, gastric juice is comprised of pepsin 0.842 mg/ml, NaCl 2.0 mg/ml, pH 1.2.

5. Method of detecting allergen, comprising the steps of reacting the protein chip of claim 1 or claim 4.

6. Method of detecting allergen, comprising the steps of utilizing the secondary or tertiary antibody, which can bind to the antibody to test and also bond with the fluorescence material, to evaluate the result, after the steps of reacting the protein chip with samples according to the claim 5.

7. Method of detecting the kinds of antibodies related with allergic reactions induced by particular allergen, comprising the steps of reacting the protein chip of the claim 1 and claim 4 with samples.

8. Method of detecting the kinds of antibodies related with allergic reaction induced by particular allergen, according to the claim 7, comprising the steps of using the secondary or tertiary antibody, which can bind to the antibody for test and also bond with the fluorescence material, to evaluate the result, after the steps of reacting the protein chip with samples.

9. Method of detecting the kinds of antibodies related with allergic reaction induced by particular allergen, according to the claim 8, wherein said fluorescence material used for evaluation purposes are different depending on each, testing antibody.
